(19) United States
(12) Patent Application Publication
(10) Pub. No.: US 2023/0329627 A1
(43) Pub. Date: Oct. 19, 2023

(54) USER INTERFACE PROVIDING DEVICE AND METHOD FOR SUPPORTING COGNITIVE ACTIVITY

(71) Applicant: LOOXID LABS INC., Daejeon (KR)

(72) Inventor: Yong Wook CHAE, Seoul (KR)

(73) Assignee: LOOXID LABS INC., Daejeon (KR)

(21) Appl. No.: 17/777,258

(22) PCT Filed: Oct. 6, 2021
(86) PCT No.: PCT/KR2021/013676

§ 371 Date: May 16, 2022

(30) Foreign Application Priority Data
Oct. 8, 2020 (KR) 10-2020-0130304

(51) Int. Cl. A61B 5/00

(52) U.S. Cl. CPC A61B 5/4088; A61B 5/6803; A61M 2205/507

(57) ABSTRACT

An apparatus and a method for providing a user interface to support a cognitive function activity according to an exemplary embodiment of the present invention are provided. According to an exemplary embodiment of the present invention provides a user interface providing apparatus for supporting a cognitive function activity including: a communication unit configured to transmit and receive data; a display unit configured to display data; and a control unit configured to be operably connected to the communication unit and the display unit, the control unit is configured to connect a head mounted display (HMD) device worn on a user for screen mirroring, receive a signal related to a wearing state of the user from the HMD device, determine a user's wearing state based on the received signal related to the wearing state, instruct the HMD device to display wearing guidance information for guiding the wearing of the user according to the determined user's wearing state, and display the mirrored cognitive function activity screen while performing the cognitive function activity by the HMD device.

### BACKGROUND OF THE DISCLOSURE

### Technical Field

The present invention relates to an apparatus and a method for providing a user interface to support a cognitive function activity.

### Background Art

Recently, as it enters an aging society, a proportion of the elderly population continuously increases so that the interest diseases, such as dementia, that are more likely to develop as people get older, is increasing.

Dementia refers to an acquired brain disease that the cognitive ability to remember and think gradually declines due to various causes to significantly interfere with the daily life.

When the dementia is diagnosed early, the onset is delayed or the state can be alleviated so that to diagnose the dementia early, a dementia diagnostic test for the elderly is being actively conducted.

However, recently, most of the dementia diagnostic tests for the elderly use the IT devices so that the elderly who are not familiar to use the IT devices have difficulty to use these devices. Further, even though there is a manager who takes care of the elderly, it is difficult for the manager to explain how to use the device to the elderly. Further, when the manager supports the dementia diagnostic test of the elderly, the complex and many additional tasks to record and correct the test hassle the manager.

Accordingly, there is a demand on a method for providing a user interface which allows a manager to easily and conveniently support cognitive function activities of the elderly to diagnose the dementia.

### SUMMARY OF THE DISCLOSURE

An object to be achieved by the present invention is to provide an apparatus and a method for providing a user interface to support a cognitive function activity.

Specifically, an object to be achieved by the present invention is to provide an apparatus and a method for providing a user interface to allow a manager to more easily and conveniently support a cognitive function activity of the elderly.

Objects of the present invention are not limited to the above-mentioned objects, and other objects, which are not mentioned above, may be clearly understood by those skilled in the art from the following descriptions.

In order to achieve the above-described object, an apparatus and a method for providing a user interface to support a cognitive function activity are provided.

According to an exemplary embodiment of the present invention, a user interface providing device for supporting a cognitive function activity includes a communication unit configured to transmit and receive data; a display unit configured to display data; and a control unit configured to be operably connected to the communication unit and the display unit, and the control unit is configured to connect a head-mounted display (HMD) device worn on a user for screen mirroring, receive a signal related to a wearing state of the user from the HMD device, determine a user's wearing state based on the received signal related to the wearing state, instruct the HMD device to display wearing guidance information for guiding the wearing of the user according to the determined user's wearing state, and display the mirrored cognitive function activity screen while performing the cognitive function activity by the HMD device.

According to an exemplary embodiment of the present invention, a user interface providing method performed by a user interface providing device for supporting a cognitive function activity includes the steps of: connecting a HMD device worn on a user for screen mirroring; receiving a signal related to a wearing state of the user from the HMD device; determining a user's wearing state based on the received signal related to the wearing state; instructing the HMD device to display wearing guidance information for guiding the wearing of the user based on the determined user's wearing state; and displaying a mirrored cognitive function activity screen while performing the cognitive function activity by the HMD device.

Other detailed matters of the exemplary embodiments are included in the detailed description and the drawings.

According to the present invention, a user who is less familiar to the IT device smoothly performs the cognitive function activity and a manager may more easily and conveniently help the progress of the cognitive function activity.

Further, according to the present invention, the hassle of the manager to record or modify the answer for the cognitive function activity is minimized and the manager may more easily explain to conduct the cognitive function activity.

Further, according to the present invention, it is possible to access a cognitive function activity more easily for dementia diagnosis and increase the professionalism and quality for the diagnosis result.

Further, according to the present invention, a cost for performing the cognitive function activity is reduced to enable the consistent cognitive function activity and individuals may easily and conveniently manage.

According to the present invention, a user customized cognitive function activity is provided to enable the personalized management according to the cognitive impairment level.

Further, according to the present invention, when the user finishes the cognitive function activity, the user feels a high sense of achievement or accomplishment and feels pleasure such as playing games with a plurality of users.

The effects according to the present invention are not limited to the contents exemplified above, and more various effects are included in the present specification.

### BRIEF DESCRIPTION OF THE DRAWINGS

FIG. 1 is a schematic view for explaining a cognitive evaluation service system to provide a user interface to support a cognitive function activity according to an exemplary embodiment of the present invention.

FIG. 2 is a schematic block diagram of a HMD device according to an exemplary embodiment of the present invention.

FIG. 3 is a schematic block diagram of a manager device according to an exemplary embodiment of the present invention.

FIG. 4 is a schematic block diagram of a service providing server according to an exemplary embodiment of the present invention.

FIG. 5 is a flowchart for explaining a method for providing a user interface to support a cognitive function activity in a manager device according to an exemplary embodiment of the present invention.

FIGS. 6 to 18 are exemplary views illustrating various interface screens related to a cognitive function test according to an exemplary embodiment of the present invention.

### DETAILED DESCRIPTION OF THE DISCLOSURE

Advantages and characteristics of the present invention and a method of achieving the advantages and characteristics will be clear by referring to exemplary embodiments described below in detail together with the accompanying drawings. However, the present invention is not limited to the following exemplary embodiments but may be implemented in various different forms. The exemplary embodiments are provided only to complete the disclosure of the present invention and to fully provide a person having ordinary skill in the art to which the present invention pertains with the category of the disclosure, and the present invention will be defined by the appended claims. With regard to the description of drawings, like reference numerals denote like components.

In this specification, the terms “have”, “may have”, “include”, or “may include” represent the presence of the characteristic (for example, a numerical value, a function, an operation, or a component such as a part”), but do not exclude the presence of additional characteristic.

In the specification, the terms “A or B”, “at least one of A or/and B”, or “at least one or more of A or/and B” may include all possible combinations of enumerated items. For example, the terms “A or B”, “at least one of A or/and B”, or “at least one or more of A or/and B” may refer to an example which includes (1) at least one A, (2) at least one B, or (3) all at least one A and at least one B.

Although the terms “first”, “second”, and the like, may be used herein to describe various components regardless of an order and/or importance, the components are not limited by these terms. These terms are only used to distinguish one component from another. For example, a first user device and a second user device may refer to different user devices regardless of the order or the importance. For example, without departing from the scope of the present invention, a first component may be referred to as a second component, and similarly, a second component may be referred to as a first component.

When a component (for example, a first component) is referred to as being “operatively or communicatively coupled with/to” or “connected to” another component (for example, a second component), it can be understood that the component is directly connected to the other element, or connected to the other element via another component (for example, a third component). In contrast, when a component (for example, a first component) is referred to as being “directly coupled with/to” or “connected to” another component (for example, a second component), it is understood that there may be another component (for example, a third component) between the components.

The terms “configured to (or set to)” may be exchangeably used with “suitable for”, “having the capacity to”, “designed to”, “adapted to”, “made to”, or “capable of” depending on the situation. The terms “configured to (or set)” may not necessarily mean only “specifically designed to” in a hardware manner. Instead, in some situation, the terms “a device configured to” may mean that the device is capable of something together with another device or components”. For example, the terms “a processor configured (or set) to perform A, B, and C” may refer to a dedicated processor (for example, an embedded processor) configured to perform the corresponding operation or a generic-purpose processor (for example, a CPU or an application processor) which is capable of perform the operations by executing one or more software programs stored in a memory device.

The terms used in this specification are merely used to describe a specific embodiment, but do not intend to limit the scope of another embodiment. A singular form may include a plural form if there is no clearly opposite meaning in the context. Terms used herein including technical or scientific terms may have the same meaning as commonly understood by those skilled in the art. Among the terms used in this specification, terms defined in the general dictionary may be interpreted as having the same or similar meaning as the meaning in the context of the related art, but is not ideally or excessively interpreted to have formal meanings unless clearly defined in this specification. In some cases, even though the terms are defined in this specification, the terms are not interpreted to exclude the embodiments of the present specification.

The features of various embodiments of the present invention can be partially or entirely bonded to or combined with each other and can be interlocked and operated in technically various ways understood by those skilled in the art, and the embodiments can be carried out independently of or in association with each other.

Hereinafter, various exemplary embodiments of the present invention will be described in detail with reference to accompanying drawings.

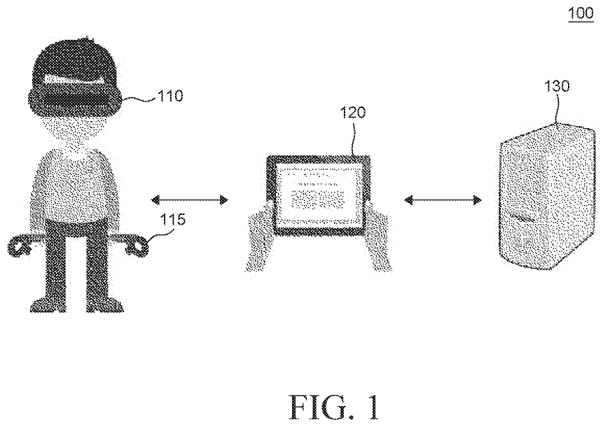

FIG. 1 is a schematic view for explaining a cognitive evaluation service system to provide a user interface to support a cognitive function activity according to an exemplary embodiment of the present invention.

Referring to FIG. 1, the cognitive function activity service system 100 may be a system providing various services for a cognitive function activity of a user. Here, the cognitive function activity may include performing a test for evaluating a cognitive function and/or providing of a cognitive function training content for improving a cognitive function (or ability) of the user according to a cognitive function evaluation result.

The cognitive function activity service system 100 includes a head-mounted display (HMD) device 110 acquiring biometric data of the user and providing contents for a cognitive function test, a manager device 120 for a manager who guides (or supervises or supports) a cognitive function test of the user, and a service providing server 130 providing diagnostic result data based on the user's biometric data and the cognitive function test. Here, the cognitive function activity service system 100 determines a cognitive state of the user by means of the cognitive function test. When it is determined that the cognitive function of the user is deteriorated, the cognitive function activity service system 100 may determine whether dementia corresponding to the cognitive impairment such as mild cognitive impairment, Alzheimer, and/or Parkinson's disease and pseudodementia such as a depression occurs. According to various exemplary embodiments, the cognitive function activity service system 100 may provide a cognitive function training content according to the cognitive function evaluation result. For example, the cognitive function training content may be a training content determining whether the user has a cognitive impartment based on the cognitive function evaluation result. Or the cognitive function training content for improving the cognitive function of the user suitable for the level of cognitive impairment.

First, the HMD device 110 may be mounted on a head of the user and provide a virtual reality image for a cognitive function to the user to allow the user to have a spatial and temporal experiences similar to the reality. At the same time, the HMD device 100 may be a complex virtual experience device capable of sensing physical, cognitive, and emotional changes of the user who is undergoing a virtual experience by acquiring biosignal of the user, such as a gaze and a brainwave.

Specifically, the HMD device 110 is formed to be wearable on a head of the user. The HMD device 110 may be implemented to perform the image processing for virtual reality, augmented reality, or mixed reality (VR, AR, or MR) in the HMD device 100. Otherwise, The HMD device 110 may be partially equipped an image output device (not illustrated) that provides a virtual, augmented, or mixed reality image, and the image processing for the virtual, augmented, or mixed reality is performed in the mounted image output device.

At least one sensor module (not illustrated) acquiring gaze data and brainwave data of the user may be formed at one side of the HMD device 110. At least one sensor module may include at least one of a gaze sensor module measuring a user's gaze and a brainwave sensor module measuring a user's brainwave. For example, the gaze sensor module may be a camera module that photographs an image including a pupil and the brainwave sensor module may be an electroencephalogram (EEG) sensor module, but are not limited thereto. According to various exemplary embodiments, at least one sensor module may be installed in the HMD device 110 or may be implemented as a separate device.

The HMD device 110 may be connected to the manager device 120 for screen mirroring. The HMD device 110 may transmit screen mirroring data to the manager device 120. Here, the screen mirroring data may refer to screen data displayed on a display unit of the HMD device 110 or screen data displayed on a display unit of the image output device.

The HMD device 110 may execute virtual reality contents for a cognitive function activity. The HMD device 110 may transmit various data acquired with regard to the executed virtual reality contents to the manager device 120 or the service providing server 130. For example, the virtual reality content for the cognitive function activity may include a virtual reality based cognitive function test program (or an application) that is conducted in the order of device connection, communication connection, tutorial, cognitive function activity and/or analysis result providing. For another example, the virtual reality content for the cognitive function activity may include a cognitive function training program (or an application) for improving a cognitive function of the user. Here, the tutorial and the cognitive function test may be implemented in the form of a game, but are not limited thereto. According to various exemplary embodiments, the cognitive function training program is also implemented in the form of a game, but is not limited thereto. The cognitive function training content may be appropriately provided to the user depending on whether the user has a cognitive impairment, a cognitive impairment level, and/or a type of a cognitive impairment.

Specifically, the HMD device 110 may transmit a signal related to a device connection state and/or a communication connection state to the manager device 120. Here, the signal related to the device connection state may be a signal related to at least one of abnormality of a program, a connection (or operation) state with the sensor module 210, a power state of the HMD device 110, a wearing state of the HMD device 110, a charging state of the HMD device 110, and a connection state with the input device 115. The signal related to the communication connection state may be a signal related to at least one of a power state of communication connection equipment that connects the communication with the manager device 120 and the service providing server 130 and a communication connection state with the communication connection equipment, the manager device 120, and the service providing server 130.

The HMD device 110 may transmit a biosignal acquired with regard to the cognitive function activity and input data acquired while performing the cognitive function activity to the manager device 120 or the service providing server 130. For example, the user may perform input or selection with regard to the tutorial or the cognitive function test by means of an input device (for example, a VR controller, etc.) 115 connected to the HMD device 110. Here, the bio signal may be biosignal related to the input data acquired by the input device 115.

Further, the HMD device 110 may display various guidance information according to an instruction from the manager device 120. Specifically, the HMD device 110 may receive an instruction for displaying guidance information guiding device connection and/or communication connection from the manager device 120. The HMD device 110 may display guidance information for the device connection and/or the communication connection according to the instruction.

According to various exemplary embodiments, the HMD device 110 may receive instruction for conducting the cognitive function activity from the manager device 120. The HMD device 110 may conduct the cognitive function activity according to the instruction.

According to various exemplary embodiments, the HMD device 110 may receive an instruction for displaying guidance information guiding a connection state (or an operation state) of the sensor module while performing the cognitive function activity from the manager device 120. Here, the connection state (or an operation state) of the sensor module may be determined according to the biosignal obtained from the HMD device 110. Thereafter, the HMD device 110 may display the guidance information according to the instruction.

According to various exemplary embodiments, the HMD device 110 may receive evaluation result data for the cognitive function activity from the manager device 120 or the service providing server 130. The HMD device 110 may display an analysis result screen representing the received evaluation result data.

Next, the manager device 120 is an electronic device providing a user interface to support the cognitive function activity of the user. The manager device 120 may include at least one of a smart phone, a tablet PC (Personal computer), a notebook computer and/or a personal computer therefor.

The manager device 120 may perform communication connection with the HMD device 110 for screen mirroring. When the manager device is connected to the HMD device 110, the manager device 120 may receive screen mirroring data from the HMD device 110 to display the received screen mirroring data on the display unit of the manager device 120. By doing this, the manager may support the device connection, communication connection, tutorial conduction, cognitive function test, providing of cognitive function training content, and providing of an analysis result while sharing the screen of the HMD device 110 worn on the user using the manager device 120.

The manager device 120 may receive various data acquired with regard to the cognitive function activity from the HMD device 110. The manager device 120 may transmit an instruction for displaying various guidance information related to the cognitive function activity to the HMD device 110 based on the received various data.

Specifically, the manager device 120 may receive a signal related to a device connection state and/or a communication connection state from the HMD device 110. The manager device 120 may transmit an instruction for displaying guidance information guiding device connection and/or communication connection to the HMD device 110 based on the received signal.

For example, the manager device 120 may receive a signal related to at least one of abnormality of a program, a connection (or operation) state to the sensor module 210, a power state of the HMD device 110, a wearing state of the HMD device 110, a charging state of the HMD device 110, and a connection state with the input device 115. The manager device 120 may transmit an instruction for displaying guidance information guiding at least one of execution of the program, connection (or operation) to the sensor module 210, power on/off of the HMD device 110, wearing of the HMD device 110, charging of the HMD device 110, and connection to the input device 115 to the HMD device 110 based on the received signal.

According to various exemplary embodiments, the manager device 120 may receive a signal related to at least one of the power state of the communication connection equipment and the communication connection state. The manager device 120 may transmit an instruction for displaying guidance information guiding at least one of power on/off of the communication connection equipment and communication connection to the HMD device 110 based on the received signal.

According to various exemplary embodiments, when the device connection and the communication connection are completed, the manager device 120 may transmit an instruction for conducting the cognitive function activity to the HMD device 110.

According to various exemplary embodiments, the manager device 120 may receive a biosignal from at least one sensor module installed in the HMD device 110 or at least one sensor that is separately implemented, with regard to the cognitive function activity. The manager device 120 may determine whether the received biosignal is an analyzable signal. When the biosignal is a non-analyzable signal, The manager device 120 may transmit an instruction for displaying guidance information guiding a connection state to a sensor module or a separate sensor to the HMD device 110.

According to various exemplary embodiments, when the cognitive function activity is finished, the manager device 120 may display a mirrored analysis result screen representing evaluation result data of the cognitive function activity. Here, the mirrored analysis result screen may include evaluation result data based on a subject (for example, a lifestyle manager, a doctor, or the like) who supports the cognitive function activity.

The evaluation result data may be generated by the service providing server 130 based on the biosignal acquired from the HMD device 110 and the input data. According to various exemplary embodiments, the evaluation result data may be generated by the manager device 120.

Next, the service providing server 130 may include a general purpose computer, a laptop computer, and/or a cloud server performing various arithmetic operations to provide a service regarding the cognitive function activity.

Specifically, the service providing server 130 may receive a biosignal and input data from the HMD device 110 or the manager device 120. The service providing server 130 may generate evaluation result data for the cognitive function activity based on the received biosignal and the input data.

In order to generate the evaluation result data, the service providing server 130 may use an artificial intelligence-based prediction model trained to predict the cognitive ability of the user by inputting the biosignal and the input data.

The service providing server 130 may predict the cognitive ability of the subject by inputting the biosignal and the input data using the evaluation model. The service providing server 130 may generate the evaluation result data for the predicted cognitive ability. The service providing server 130 may transmit the generated evaluation result data to the manager device 120.

According to various exemplary embodiments, the service providing server 130 may generate analysis result data by means of various statistical analysis. The service providing server 130 may transmit the explainable feature data trained by the artificial intelligence-based prediction model and the generated analysis result data to the HMD device 110 and/or the manager device 120.

As described above, according to the present invention, the manager may help a user who has difficulty to use the device to smoothly perform the cognitive function test.

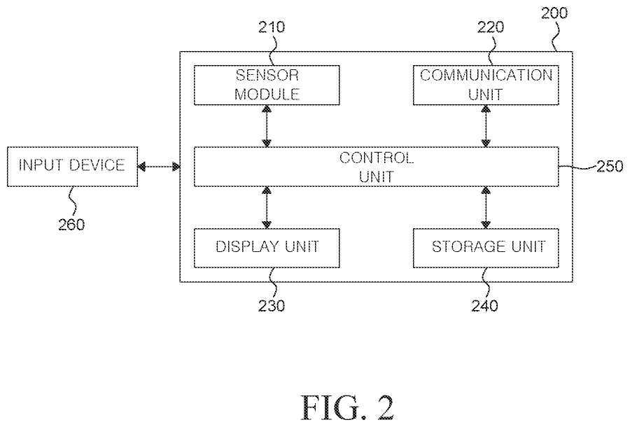

Hereinafter, Each component constituting the cognitive function activity service system 100, the HMD device 110, the manager device 120, and the service providing server 130 will be described in detail with reference to FIGS. 2, 3, and 4. The proposed exemplary embodiment explains a case that the user performs the cognitive function test among the cognitive function activities.

Hereinafter, the HMD device 110 will be described in more detail with reference to FIG. 2.

FIG. 2 is a schematic block diagram of a HMD device according to an exemplary embodiment of the present invention.

Referring to FIG. 2, the HMD device 200 includes a sensor module 210, a communication unit 220, a display unit 230, a storage unit 240, and a control unit 250. In the proposed exemplary embodiment, the HMD device 200 refers to the HMD device 110 of FIG. 1.

The sensor module 210 may measure a biosignal of the user. One or more sensor modules 210 may be provided and for example, the sensor module may include at least one of a gaze sensor module which measures a gaze of the user and a brainwave sensor module which measures a brainwave of the user. According to various exemplary embodiments, the sensor module 210 may be installed in the HMD device 200 or may be implemented as a separate device. For example, the gaze sensor module is installed in the HMD device 200 and the brainwave sensor module may be implemented as a separate device which is attachable to the head of the user, but is not limited thereto.

The communication unit 220 may connect the HMD device 200 so as to communicate with the external device. The communication unit 220 is connected to the manager device 120 or the service providing server 130 using wired/wireless communication to transmit and receive various data regarding the cognitive function test.

Specifically, the communication unit 220 may transmit screen mirroring data to the manager device 120 connected for the screen mirroring. The communication unit 220 may transmit a signal related to the device connection and/or the communication connection to the manager device 120. The communication unit 220 may receive instructions for displaying the guidance information from the manager device 120.

According to various exemplary embodiments, the communication unit 220 may receive an instruction for displaying guidance information guiding the progress of the tutorial or the cognitive function test from the manager device 120. The communication unit 220 may transmit a biosignal acquired with regard to the tutorial or the cognitive function test and input data acquired during the tutorial or the cognitive function test to the manager device 120 or the service providing server 130.

According to various exemplary embodiments, the communication unit 220 may receive the evaluation result data for the cognitive function test from the manager device 120 or the service providing server 130.

The display unit 230 may display various contents (for example, texts, images, videos, icons, banners, symbols, or the like) to the user. Specifically, the display unit 230 may display various interface screens for the cognitive function test. For example, the display unit 230 may display various interface screens of a virtual reality based cognitive function test program executed for the cognitive function test.

The storage unit 240 may store various data used for the cognitive function test. For example, the storage unit 240 may store various data used to execute the virtual reality based cognitive function test program or display various interface screens of the executed cognitive function test program.

According to various embodiments, the storage unit 240 may include at least one type of storage medium of flash memory type, hard disk type, multimedia card micro type, and card type memories (for example, SD or XD memory and the like), a random access memory (RAM), a static random access memory (SRAM), a read only memory (ROM), an electrically erasable programmable read only memory (EEPROM), a programmable read only memory (PROM), a magnetic memory, a magnetic disk, and an optical disk. The HMD device 200 may operate in association with a web storage performing a storage function of the storing unit 240 on the Internet.

The control unit 250 may be operably connected to the sensor module 210, the communication unit 220, the display unit 230, the storage unit 240, and the input device 260. The control unit 250 may execute various commands for the cognitive function test. According to various exemplary embodiments, when the brainwave sensor module is included as a separate device or the gaze sensor module and the brainwave sensor module are included as a separate device, the device may be driven by the command of the control unit equipped in this device. In this case, the control unit 250 may collect or store the data received from a separate device or transmits the data to the service providing server 130.

The control unit 250 may perform the connection for screen mirroring with the manager device 120 and may transmit the screen mirroring data to the manager device 120.

The control unit 250 may execute the virtual reality based cognitive function test program and may display various interface screens related to the executed cognitive function test program. Screen data of the interface screen may be transmitted to the manager device 120 as screen mirroring data.

The cognitive function test program may be performed in the order of device connection, communication connection, tutorial, the cognitive function test, and analysis result providing. For example, the device connection state may refer to a procedure to check at least one of the abnormality of a program, a connection (or operation) state to the sensor module 210, a power state of the HMD device 200, a wearing state of the HMD device 200, a charging state of the HMD device 200, and a connection state to the input device 260. Further, the device connection state may refer to a procedure for provide guidance information therefor. The communication connection may refer to a procedure to check at least one of a power state of communication connection equipment which connects the communication with the manager device 120 and the service providing server 130 and a communication connection state with the communication connection equipment, the manager device 120, and the service providing server 130. Further, The communication connection may refer to a procedure for provide guidance information therefor.

Specifically, the control unit 250 may display an interface screen related to the device connection and/or the communication connection. The control unit 250 may transmit a signal related to the device connection state and/or the communication connection state to the manager device 120. For example, the control unit 250 may transmit a signal related to at least one of abnormality of a program, a connection (or operation) state to the sensor module 210, a power state of the HMD device 110, a wearing state of the HMD device 200, a charging state of the HMD device 200, and a connection state to the input device 260 to the manager device 120. According to various exemplary embodiments, the control unit 250 may transmit the signal related to at least one of a power state of communication connection equipment connecting the communication with the manager device 120 and the service providing server 130 and a communication connection state with the communication connection equipment, the manager device 120, and the service providing server 130 to the manager device 120.

When an instruction for displaying guidance information guiding the device connection and/or the communication connection is received from the manager device 120, the control unit 250 may display an interface screen including the guidance information. For example, the control unit 250 may display the guidance information to appear on a partial area of the interface screen, but is not limited thereto. The control unit 250 may display the guidance information to appear in various methods.

Specifically, the control unit 250 may receive an instruction for displaying guidance information guiding at least one of execution of the program, connection (or operation) to the sensor module 210, power on/off of the HMD device 200, wearing of the HMD device 200, charging of the HMD device 200, and connection to the input device 260. The control unit 250 may display the interface screen including the guidance information according to the instruction. According to various exemplary embodiments, the control unit 250 may receive an instruction for displaying guidance information guiding at least one of power on/off of the communication connection equipment and communication connection. The control unit 250 may display the interface screen including the guidance information according to the instruction.

According to various exemplary embodiments, when the device connection and the communication connection are completed, the control unit 250 may receive the instruction for conducting the tutorial or the cognitive function test from the manager device 120. The control unit 250 may display an interface screen for the tutorial or the cognitive function test according to the instruction.

According to various exemplary embodiments, the control unit 250 may receive an instruction for displaying guidance information guiding a connection state of the sensor module determined according to a biosignal acquired during the tutorial or the cognitive function test from the manager device 120. The control unit 250 may display an interface screen including the guidance information according to the instruction.

According to various exemplary embodiments, the control unit 250 may receive evaluation result data for the cognitive function test from the manager device 120 or the service providing server 130. The control unit 250 may display an interface screen representing the received evaluation result data.

The above-described operations may be proceeded in the described order, or a reverse order, or simultaneously performed, but is not limited thereto.

Finally, the input device 260 is connected to the HMD device 200 and acquires the user's input data related to the cognitive function test, and transmits the acquired input data to the HMD device 200. For example, the input device 260 may be a VR controller, but is not limited thereto. Various input devices connected to the HMD device to acquire input data may be used.

As described above, according to the present invention, even though the user is less familiar to the device, the user may easily and conveniently perform the cognitive function test with the help of the manager.

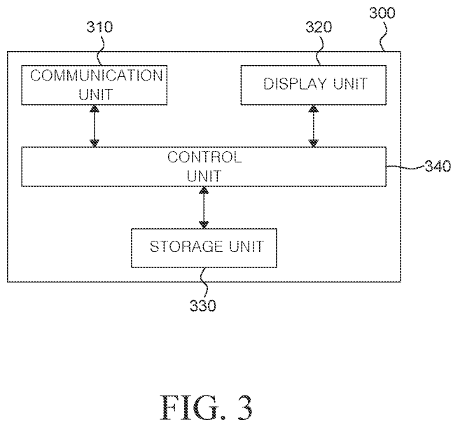

Hereinafter, the manager device 120 will be described in more detail with reference to FIG. 3.

FIG. 3 is a schematic block diagram of a manager device according to an exemplary embodiment of the present invention.

Referring to FIG. 3, the manager device 300 includes a communication unit 310, a display unit 320, a storage unit 330, and a control unit 340. In the proposed exemplary embodiment, the manager device 300 refers to the manager device 120 of FIG. 1.

The communication unit 310 may connect the manager device 300 so as to communicate with the external device. The communication unit 310 is connected to the HMD device 110 and the service providing server 130 using wired/wireless communication to transmit and receive various data to support the cognitive function test. Specifically, the communication unit 310 may receive screen mirroring data from the HMD device 110 connected for the screen mirroring. The communication unit 310 may receive a signal related to the device connection and/or the communication connection of the HMD device 110 from the HMD device 110. The communication unit 310 may transmit guidance information therefor to the HMD device 110.

The communication unit 310 may transmit an instruction for displaying guidance information guiding to conduct the tutorial or the cognitive function test from the manager device 120 to the HMD device 110. The communication unit 310 may receive a biosignal acquired with regard to the tutorial or the cognitive function test and input data acquired during the tutorial or the cognitive function test from the HMD device 110. The communication unit 310 may transmit the guidance information therefor to the HMD device 110.

According to various exemplary embodiments, the communication unit 310 may receive evaluation result data for the cognitive function test from the service providing server 130. The communication unit 310 may transmit the received evaluation result data to the HMD device 110. According to various exemplary embodiments, when the manager device 300 conducts the cognitive evaluation, the evaluation result data generated by the manager device 300 is transmitted to the HMD device 110.

The display unit 320 may display various contents to the manager. Specifically, the display unit 320 may display various interface screens to support the cognitive function test. For example, the display unit 320 may display an interface screen representing screen mirroring data received from the HMD device 110.

According to various embodiments, the display unit 320 may include a touch screen and for example, may receive touch, gesture, proximity, drag, swipe, or hovering input using an electronic pen or a part of a body of the user.

The storage unit 330 may store various data used to support the cognitive function test. For example, the storage unit 330 may store biometric data, data regarding various events (for example, face motion, controller manipulation) generated by the user while executing the program, and data regarding an event (for example, program start and end) generated by the program.

In various embodiments, the storage unit 330 may include at least one storage medium of a flash memory type, a hard disk type, a multimedia card micro type, a card type memory (for example, an SD or XD memory, etc.), a RAM, an SRAM, a ROM, an EEPROM, a PROM, a magnetic memory, a magnetic disk, and an optical disk. The manager device 300 may operate in association with a web storage which performs the storing function of the storage unit 330 on the Internet.

The control unit 340 may be operatively connected to the communication unit 310, the display unit 320, and the storage unit 330. The control unit 340 may perform various commands to provide a user interface to support the cognitive function test.

Specifically, the control unit 340 may perform communication connection with the HMD device 110 for screen mirroring. When the control unit is connected to the HMD device 110, the control unit 340 may receive screen mirroring data from the HMD device 110 to display the received screen mirroring data on the display unit 320.

The control unit 340 may receive a signal related to a device connection state and/or a communication connection state from the HMD device 110. The control unit 340 may transmit an instruction for displaying guidance information guiding the device connection and/or communication connection to the HMD device 110 based on the received signal.

According to an exemplary embodiment, the control unit 340 may receive a signal related to the abnormality of the program from the HMD device 110. Here, the signal related to the abnormality of the program may be a signal indicating whether a program for the cognitive function test normally executes.

The control unit 340 may determine the abnormality of the program based on the received signal. Accordingly, The control unit 340 may transmit an instruction for displaying the guidance information guiding the execution of the program to the HMD device 110. For example, when it is determined that the program is abnormal, the control unit 340 may transmit the instruction for displaying the guidance information to the HMD device 110. Here, the guidance information guiding the execution of the program may be information for guiding re-execution of the program, but is not limited thereto.

According to various exemplary embodiments, the control unit 340 may receive a signal related to a connection state with a sensor module of the HMD device 110 or a separate sensor device from the HMD device 110. Here, the signal related to the connection state may be a signal indicating whether the sensor module or a separate sensor device is normally connected or operates.

The control unit 340 may determine a connection (or operation) state with the sensor module 210 or the separate sensor device based on the received signal. The control unit 340 may transmit an instruction for displaying guidance information guiding the connection (or the operation) with the sensor module 210 according to the determined connection state to the HMD device 110. For example, when the connection state with the sensor module 210 or the separate sensor device is not normal, the control unit 340 may transmit an instruction for displaying guidance information to the HMD device 110. Here, the guidance information guiding the connection with the sensor module 210 may be information guiding to confirm whether the sensor module is normally attached or the power is turned on, but is not limited thereto.

According to various exemplary embodiments, the control unit 340 may receive a signal related to the power state of the HMD device 110 from the HMD device 110. Here, the signal related to the power state of the HMD device 110 may be a signal indicating whether the power of the HMD device 110 is turned on.

The control unit 340 may determine a power state of the HMD device 110 based on the received signal. The control unit 340 may transmit an instruction for displaying guidance information guiding the power on/off of the HMD device 110 according to the determined power state to the HMD device 110. For example, when the power of the HMD device 110 is turned off, the control unit 340 may transmit an instruction for displaying guidance information to the HMD device 110. Here, the guidance information guiding the power on/off of the HMD device 110 may be information for guiding a method for turning on/off the power of the HMD device 110 or a method for checking a power LED, but is not limited thereto.

According to various exemplary embodiments, the control unit 340 may receive a signal related to the user's wearing state of the HMD device 110 from the HMD device 110. Here, the signal related to the wearing state may be a signal indicating that the wearing state of the user is a wearing state suitable for conducting the cognitive function test.

The control unit 340 may determine the user's wearing state based on the received signal. The control unit 340 may transmit an instruction for displaying the guidance information guiding the wearing of the user according to the determined wearing state to the HMD device 110. For example, when the user's wearing state is not suitable for conducting the cognitive function test, the control unit 340 may transmit an instruction for displaying guidance information to the HMD device 110. Here, the guidance information guiding the wearing of the user may be information for guiding re-wearing of the HMD device 110, but is not limited thereto.

According to various exemplary embodiments, the control unit 340 may receive a signal related to the charging state of the HMD device 110 from the HMD device 110. Here, the signal related to the charging state may be a signal indicating that the charging state of the HMD state 110 is a charging state suitable for conducting the cognitive function test.

The control unit 340 may determine a charging state of the HMD device 110 based on the received signal. The control unit 340 may transmit an instruction for displaying guidance information guiding the charging of the HMD device 110 according to the determined charging state to the HMD device 110. For example, when the charging state of the HMD device 110 is below a predetermined threshold charging percentage, the control unit 340 may transmit an instruction for displaying guidance information to the HMD device 110. Here, the guidance information guiding the charging may be information for guiding to charge the HMD device 110, but is not limited thereto.

According to various exemplary embodiments, the control unit 340 may receive a signal related to the connection state of the input device 115 from the HMD device 110. Here, the signal related to the connection state of the input device 115 may be a signal indicating whether the input device 115 is normally connected.

The control unit 340 may determine a connection state of the input device 115 based on the received signal. The control unit 340 may transmit an instruction for displaying guidance information guiding the connection of the input device 115 according to the determined connection state to the HMD device 110. For example, when the input device 115 is not connected, the control unit 340 may transmit an instruction for displaying guidance information to the HMD device 110. Here, the guidance information guiding the connection of the input device 115 may be information for guiding a method for turning on/off the power of the input device 115 or a method for checking a power LED, but is not limited thereto.

According to various exemplary embodiments, the control unit 340 may receive a signal related to the power state of the communication connection equipment from the HMD device 110. Here, the signal related to the power state of the communication connection equipment may be a signal indicating whether a power of the communication connection equipment is turned on. For example, when the communication connection equipment for communication connection is not searched, the control unit 340 may determine that the power of the communication connection equipment is turned off.

The control unit 340 may determine a power state of the communication connection equipment based on the received signal. The control unit 340 may transmit an instruction for displaying guidance information guiding the power on/off of the communication connection equipment according to the determined power state to the HMD device 110. For example, when the power of the communication connection equipment is turned off, the control unit 340 may transmit the instruction for displaying the guidance information to the HMD device 110. Here, the guidance information guiding the power on/off of the communication connection equipment may be information guiding a method for turning on/off the power of the communication connection equipment, a method for checking a power LED, or a method for charging the communication connection equipment, but is not limited thereto.

According to various exemplary embodiments, the control unit 340 may receive a signal related to the communication connection state of the communication connection equipment from the HMD device 110. Here, the signal related to the communication connection state of the communication connection equipment may be a signal indicating a communication connection of the communication connection equipment.

The control unit 340 may determine a communication connection state of the communication connection equipment based on the received signal. The control unit 340 may transmit an instruction for displaying guidance information guiding the communication connection of the communication connection equipment according to the communication connection state to the HMD device 110. For example, when the communication connection speed is lower than a predetermined threshold speed or the communication connection is disconnected, the control unit 340 may transmit the instruction for displaying the guidance information to the HMD device 110. Here, the guidance information guiding the communication connection may be information guiding a method for turning on/off the power of the communication connection equipment or a method for connecting to another communication connection equipment, but is not limited thereto.

According to various exemplary embodiments, when the device connection and the communication connection are completed, the control unit 340 may transmit an instruction for displaying guidance information guiding the progress of the tutorial or the cognitive function test to the HMD device 110.

According to various exemplary embodiments, the control unit 340 may receive a biosignal and/or input data from the HMD device 110 while the HMD device 110 conducts the tutorial. The control unit 340 may determine whether the received biosignal is an analyzable signal. If the biosignal is a non-analyzable signal, The control unit 340 may transmit an instruction for displaying guidance information guiding connection to the sensor module of the HMD device 110 or a separate sensor device to the HMD device 110.

In order to determine whether the biosignal is analyzable, the control unit 340 may determine whether the received biosignal includes at least one peak. Otherwise, when at least one peak is not included, the control unit 340 may determine that the biosignal is a non-analyzable signal.

According to various exemplary embodiments, in order to determine whether the biosignal is analyzable, the control unit 340 may use an artificial intelligence-based classification model trained to classify whether the biosignal is an analyzable signal or a non-analyzable signal by inputting the biosignal. For example, the classification model may be a model based on a deep learning algorithm. The deep learning algorithm may be at least one of a deep neural network (DNN), a convolutional neural network (CNN), a deep convolution neural network (DCNN), a recurrent neural network (RNN), a restricted Boltzmann machine (RBM), a deep belief network (DBN), and a single shot detector (SSD), but is not limited thereto. The deep learning algorithm may be based on various algorithms learning a reference signal suitable to be analyzed and classify an analyzable signal or a non-analyzable signal, based on a new biosignal.

According to various exemplary embodiments, the control unit 340 may display an interface screen for supporting the conducting of the tutorial while executing the tutorial. For example, the interface screen may further include information indicating a charging state of the HMD device 110, a charging state of the input device 115, whether to execute the tutorial, a tutorial conducting time, a frame rate of the HMD device screen, a sampling rate of the brainwave signal, and a sampling rate of the gaze signal, and a graphic object to stop or restart the tutorial, together with the mirrored tutorial screen of the HMD device 110, but is not limited thereto.

According to various exemplary embodiments, when the tutorial is finished, the control unit 340 may transmit an instruction for conducting the cognitive function test to the HMD device 110.

According to various exemplary embodiments, the control unit 340 may display an interface screen for monitoring the cognitive function test while conducting the cognitive function test by the HMD device. For example, the interface screen may further include information indicating a charging state of the HMD device 110, a charging state of the input device 115, whether to execute the cognitive function test, a conducting time of the cognitive function test, a frame rate of the HMD device screen, a sampling rate of the brainwave signal, and a sampling rate of the gaze signal, together with mirrored cognitive function test screen of the HMD device 110, but is not limited thereto.

According to various exemplary embodiments, when the cognitive function test is completed, the control unit 340 may determine whether the biosignal acquired from the HMD device 110 is an analyzable signal, during the cognitive function test. When the biosignal is a non-analyzable signal, the control unit 340 may transmit an instruction for displaying guidance information guiding the connection of the HMD device 110 with the sensor module or the separate sensor device to the HMD device 110. According to various exemplary embodiments, the control unit 340 may transmit an instruction for re-conducting the cognitive function test to the HMD device 110.

According to various exemplary embodiments, the control unit 340 may transmit the biosignal and the input data received from the HMD device 110 during the cognitive function test to the service providing server 130.

According to various exemplary embodiments, when the cognitive function test is completed, the control unit 340 may receive evaluation result data for the cognitive function test from the service providing server 130. The control unit 340 may display an interface screen representing the received evaluation result data. The evaluation result data may be provided based on the manager. Different evaluation result data may be provided depending on whether the manager is a lifestyle manager or a doctor. For example, when a subject who supports the cognitive function test is a lifestyle manager, the evaluation result data may include a relative score for every cognitive function test, group average comparison data, score ranking data in a user group, and a score distribution and/or percentage data for every cognitive function test. According to various exemplary embodiments, when a subject who supports the cognitive function test is a doctor, the evaluation result data may include replay screen data for the cognitive function test, a score and brainwave index changing trend data, left/right brain activity correlation coefficient data, cognitive/exercise/consumed time analysis data, reaction time distribution data of mainly used/assistant hand and/or performance change data according to a time, but is not limited thereto.

According to various exemplary embodiments, the control unit 340 may display the mirrored analysis result screen of the HMD device.

According to various exemplary embodiments, when the cognitive function test is completed, the control unit 340 may generate evaluation result data for the cognitive function test based on the biometric information and input data. Specifically, the control unit 340 may predict a cognitive ability of the user using a prediction model trained to predict the cognitive ability of the user by inputting the biosignal and the input data. The control unit 340 may generate evaluation result data for the predicted cognitive ability.

The control unit 340 may transmit the evaluation result data to the HMD device 110 and may display an interface screen indicating the evaluation result data. According to various exemplary embodiments, the control unit 340 may display the mirrored analysis result screen.

As described above, according to the present invention, a manager supports a user who has a difficulty to conduct the cognitive function test to smoothly conduct the cognitive function test. And a time and a resource taken to conduct the cognitive function test may be minimized.

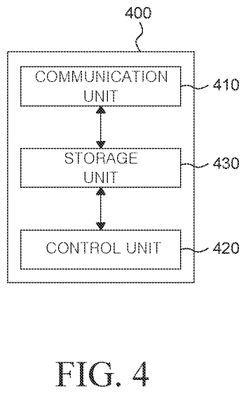

Hereinafter, the service providing server 130 will be described in more detail with reference to FIG. 4.

FIG. 4 is a schematic block diagram of a service providing server according to an exemplary embodiment of the present invention.

Referring to FIG. 4, the service providing server 400 includes a communication unit 410, a storage unit 420, and a control unit 430. In the proposed embodiment, the service providing service 400 may refer to the service providing server 130 of FIG. 1.

The communication unit 410 communicatively may connect the service providing server 400 with the external device. The communication unit 410 is connected to the HMD device 110 and the manager device 120 using wired/wireless communication to transmit and receive various data to provide a service related to the cognitive function test. Specifically, the communication unit 410 may receive the biosignal and the input signal from the HMD device 110 or the manager device 120. the communication unit 410 may transmit the evaluation result data for the cognitive function test to the HMD device 110 or the manager device 120.

The storage unit 420 may store various data used to provide a service related to the cognitive function test. In various embodiments, the storage unit 420 may include at least one storage medium of a flash memory type, a hard disk type, a multimedia card micro type, a card type memory (for example, an SD or XD memory, etc.), a RAM, an SRAM, a ROM, an EEPROM, a PROM, a magnetic memory, a magnetic disk, and an optical disk. The service providing server 400 may operate in association with a web storage which performs the storing function of the storage unit 420 on the Internet.

The control unit 430 may be operatively connected to the communication unit 410 and the storage unit 420. The control unit 430 may perform various commands to provide a service related to the cognitive function test.

Specifically, the control unit 430 may receive a biosignal and input data related to the cognitive function test from the HMD device 110 or the manager device 120. The control unit 430 may generate evaluation result data for the cognitive function test based on the received biosignal and input data.

In order to generate the evaluation result data, the control unit 430 may use an artificial intelligence-based prediction model trained to predict the cognitive ability of the user by inputting the biosignal and the input data of the user.

Such an artificial intelligence-based prediction model may use a classification model learning previously stored biometric data of the user and inferring a cognitive function state based on newly input biometric data of a new user.

For example, the prediction model or the classification model may include: acquiring first biometric data of a plurality of users; fine-tuning a prediction model based on the acquired first biometric data and a fixed learning parameter; and outputting a user state predicted using a fine-tuned prediction model by inputting second biometric data for predicting a user state for at least one user. The fixed learning parameter may be different from each other. the prediction model or the classification model is a model implemented by user state prediction method extracted based on a first model learned to predict user states for a plurality of users by inputting first biometric data for a plurality of users.

The fine-tuning may include extracting the fixed learning parameter using the first model by inputting the first biometric data and applying the fixed learning parameter to the prediction model.

The extracting the fixed learning parameter may include training the first model by inputting the first biometric data and extracting the updated learning parameter of the first model by the learning as the fixed learning parameter, from the first model.

The first model may include a plurality of layers and a second model having the same configuration as the prediction model.

The plurality of layers may include a first layer for calculating feature data using similarity data representing a similarity between the first biometric data and a predetermined learning parameter and a second layer for compressing the feature data.

The training the first model may include: calculating the similarity data by means of the first layer; calculating the feature data by performing convolution on the calculated similarity data; compressing the calculated feature data by means of the second layer; and labeling or classifying the user state for the plurality of users using the second model by inputting the compressed data.

The calculating the similarity data by means of the first layer may include: converting the first biometric data and the learning parameter into a one-dimensional vector; and calculating a cosine value between the one-dimensional vector of the first biometric data and a one-dimensional vector of the learning parameter as the similarity data.

The first layer may be a cosine similarity based convolutional layer and the second layer is a max pooling layer.

According to various exemplary embodiments, the prediction model or the classification model may be a model implemented by an emotion recognizing method. The prediction model or the classification model may include: providing contents to the user; receiving biosignal data of the user while providing the contents; and recognizing an user's emotion for the contents using an emotion classification model trained to classify an emotion based on the plurality of biosignal data labeled with the emotion. At this time, the plurality of pieces of labeled biosignal data may include first labeled biosignal data matching the emotion of the user and second labeled biosignal data having a lower labeling reliability than that of the first labeled biosignal data or does not match any emotion of the user.

The emotion classification model may be a model trained through the following steps. The emotion classification model may include: receiving at least one labeled biosignal data between first labeled biosignal data and second labeled biosignal data; encoding at least one input labeled biosignal data; decoding at least one encoded labeled biosignal data so as to acquire a reconstructed biosignal data; training a feature unit determined by the emotion classification model so as to minimize the difference between at least one input labeled biosignal data and the reconstructed biosignal data.

The emotion classification model may further include a classifying unit connected to the feature unit. The recognizing an emotion of the user for the contents may include: a first classifying step of emotion of the user for the contents based on the biosignal data of the user by means of the feature unit; and a second classifying step of emotion of the user for the contents by means of the emotion classifying unit.

Next, the control unit 430 may generate evaluation result data for the predicted cognitive evaluation and may transmit the generated evaluation result data to the HMD device 110 or the manager device 120.

According to various exemplary embodiments, the control unit 430 may generate evaluation result data based on a manager who supports the cognitive function test. For example, when a manager who supports the cognitive function test is a lifestyle manager, the control unit 430 may generate the evaluation result data including a relative score for every cognitive function test, group average comparison data, score ranking data in a user group, and a score distribution and/or percentage data for every cognitive function test. According to various exemplary embodiments, when the manager who supports the cognitive function test is a doctor, the control unit 430 may generate the evaluation result data including replay screen data for the cognitive function test, a score and brainwave index changing trend data, left/right brain activity correlation coefficient data, cognitive/exercise/consumed time analysis data, reaction time distribution data of mainly used/assistant hand and/or performance change data according to a time, but is not limited thereto.

According to various exemplary embodiments, the evaluation result data may further include brain disease diagnosis and early diagnosis assistance data such as a probability of a specific brain disease or a probability of having a specific brain disease, in addition to the above-mentioned data.

According to various exemplary embodiments, the control unit 430 may receive a biosignal from the HMD device 110 or the manager device 120. The control unit 430 may determine whether the received biosignal is analyzable. When the signal is not analyzable, the control unit may transmit an instruction for displaying guidance information guiding connection to the sensor module of the HMD device 110 or a separate sensor device to the HMD device 110 or the manager device 120.

As described above, according to the present invention, a user-customized cognitive function test result may be provided to the user.

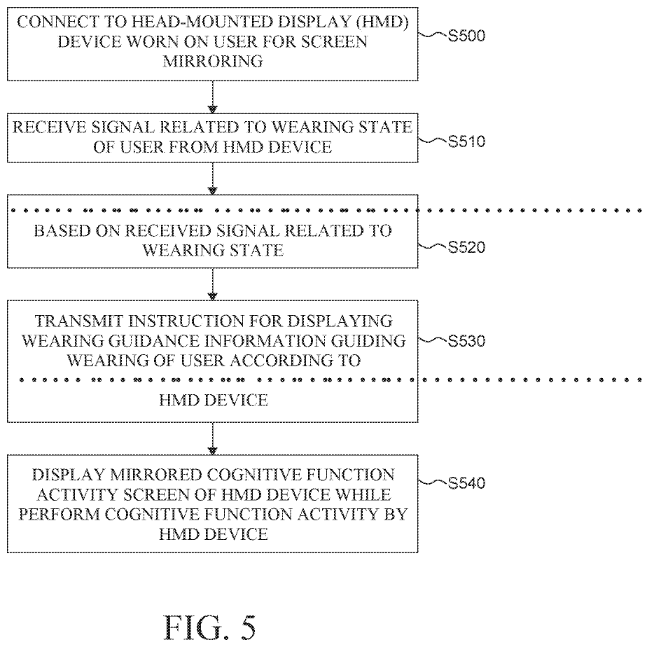

Hereinafter, a method for providing a user interface to support the cognitive function activity in the manager device 120 will be described with reference to FIG. 5.

FIG. 5 is a flowchart for explaining a method for providing a user interface to support a cognitive function activity in a manager device according to an exemplary embodiment of the present invention. Operations to be described below will be performed by the control unit 340 of the manager device 300 of FIG. 3.

Referring to FIG. 5, the manager device 300 may connect the HMD device 110 worn on the user for screen mirroring (S500) and may receive a signal related to the wearing state of the user from the HMD device 110 (S510). Here, the signal related to the wearing state may be a signal indicating the user's wearing state is a wearing state suitable for conducting the cognitive function activity.

The manager device 300 may determine the user's wearing state based on the received signal related to the wearing state (S520). For example, the manager device 300 may determine whether the user's wearing state is a wearing state suitable for conducting the cognitive function activity.

The manager device 300 may transmit an instruction for displaying wearing guidance information guiding the wearing of the user according to the determined the user's wearing state to the HMD device 110 (S530). For example, the wearing guidance information may be information for guiding re-wearing of the HMD device 110.

The manager device 300 may display the mirrored cognitive function activity screen of the HMD device 110 while performing the cognitive function activity by the HMD device 110 (S540). In other words, the manager device 300 may display an interface screen indicating screen mirroring data received from the HMD device 110. Such an interface screen may be an interface screen for monitoring the cognitive function activity of the user.

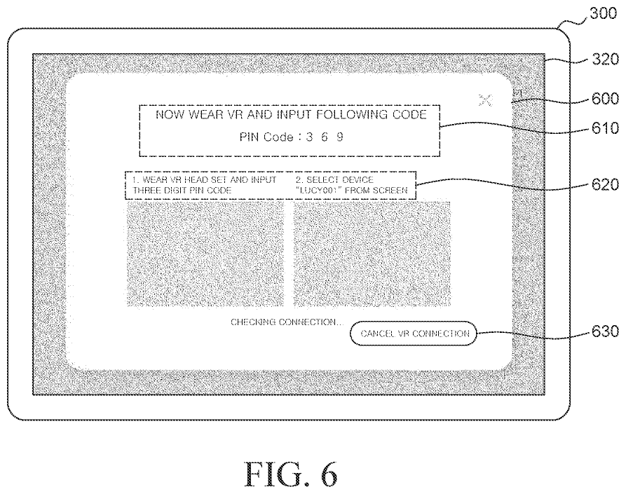

Various interface screens related to the cognitive function test among the cognitive function activities will be described with reference to FIGS. 6 to 18.

FIGS. 6 to 18 are exemplary views illustrating various interface screens related to a cognitive function test according to an exemplary embodiment of the present invention. According to the proposed exemplary embodiment, various interface screens may be displayed on the display unit 320 of the manager device 300.

Referring to FIG. 6, the manager device 300 may display an interface screen 600 for connection with the HMD device 110 for screen mirroring. Here, the interface screen 600 may include graphic objects 610 and 620 for selecting an HMD device for connection after wearing the HMD device 110 (for example, a VR headset) and inputting connection information (for example, PIN code) used to be connected with the HMD device 110. The interface screen 600 may include a graphic object (for example, VR connection cancel) 630 for cancelling the connection with the HMD device 110.

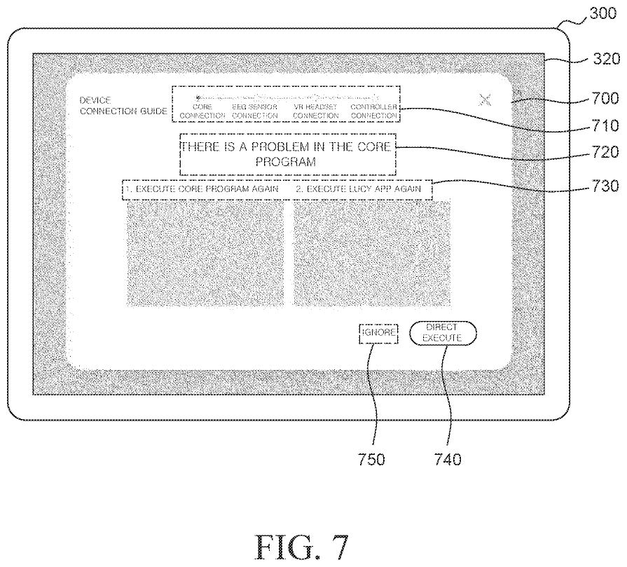

When the connection with the HMD device 110 is completed, the manager device 300 may receive the screen mirroring data from the HMD device 110 and may display the interface screen representing the received screen mirroring data. The interface screen representing the screen mirroring data will be described below in detail with reference to FIGS. 7 to 18.

The manager device 300 may receive a signal related to a device connection state and/or a communication connection state from the HMD device 110. The manager device 300 may transmit an instruction for displaying guidance information guiding device connection and/or communication connection to the HMD device 110 based on the received signal.

Specifically, the manager device 300 may receive a signal related to the abnormality of the program from the HMD device 110 and may determine whether the program is normal based on the received signal. After, The manager device 300 may transmit an instruction for displaying guidance information guiding the execution of the program to the HMD device 110 according to the determined result. Here, the program may be a program installed in the HMD device 110 and/or a program installed in the manager device 300.

Referring to FIG. 7, when the program is not normal, the manager device 300 may display a mirrored interface screen 700 including guidance information guiding the execution of the program.

Here, the mirrored interface screen 700 may include a first graphic object 710 representing a device connection process, a second graphic object 720 representing the abnormality of the program, and a third graphic object 730 representing guidance information according to the abnormality of the program. The interface screen 700 may include a fourth graphic object 740 for directly executing the program and a fifth graphic object 750 for conducting by ignoring the abnormality of the program.

The manager may support to directly execute the program or ignore the abnormality to conduct the activity by means of the interface screen.

The manager device 300 may receive a signal related to the connection state of the HMD device 110 with the sensor module or the separate sensor device. The manager device 300 may determine a connection state with the sensor module 210 or the separate sensor device based on the received signal. And then, The manager device 300 may transmit an instruction for displaying guidance information guiding the connection with the sensor module 210 according to the determined connection state to the HMD device 110.

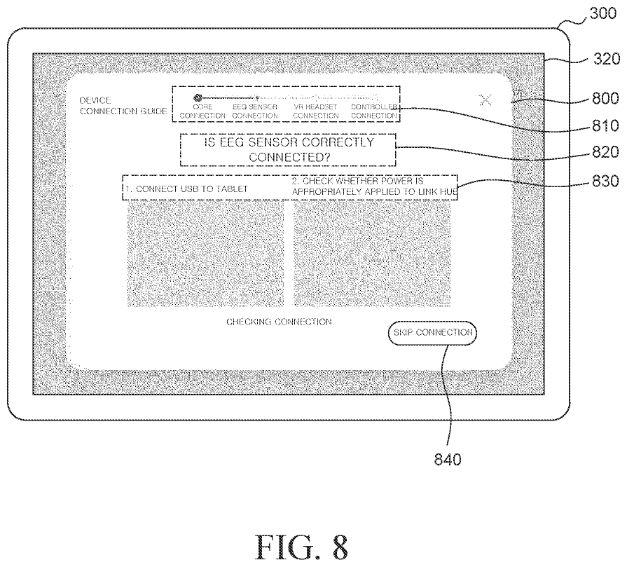

Referring to FIG. 8, the manager device 300 may check whether the sensor module or the separate sensor device is correctly connected. The manager device 300 may display a mirrored interface screen 800 including the guidance information for guiding the connection with the sensor module or the separate sensor device.

Here, the mirrored interface screen 800 may include a first graphic object 810 representing a device connection process, a second graphic object 820 for inquiring whether the sensor module or the separate sensor device is correctly connected, and a third graphic object 830 representing guidance information guiding the connection of the sensor module or the separate sensor device. Here, the first graphic object 810 may be an image or an icon representing a completed connection process or an ongoing connection process, but is not limited thereto.

According to various exemplary embodiments, the mirrored interface screen 800 may further include information for notifying that the sensor module or the separate sensor device is not normally connected. By doing this, the manager may support the connection with the sensor module or the separate sensor device using the manager device 300.

Further, the interface screen 800 may further include a fourth graphic object 840 for proceeding a next process by ignoring the sensor connection.

The manager may support the connection with the sensor module or the separate sensor device by means of the interface screen.

The manager device 300 may receive a signal related to the power state of the HMD device 110 from the HMD device 110 and may determine the power state of the HMD device 110 based on the received signal. And then, The manager device 300 may transmit the instruction for displaying guidance information guiding the power on/off of the HMD device 110 according to the determined power state to the HMD device 110.

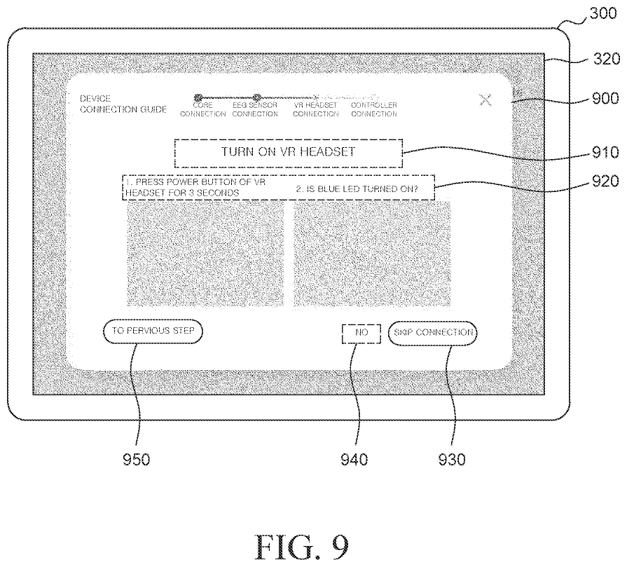

Referring to FIG. 9, the manager device 300 may display a mirrored interface screen 900 including guidance information for guiding the power on/off of the HMD device 110.

Here, the mirrored interface screen 900 may include a first graphic object 910 for instructing to turn on the HMD device 110 and a second graphic object 920 representing guidance information for guiding how to turn on the power. Further, the interface screen 900 may include a third graphic object 930 for inputting whether the power is turned on, a fourth graphic object 940 for inputting whether the power is turned off, and a fifth graphic object 950 for proceeding to a previous step.

The manager device may support to turn on/off the power of the HMD device 110 by means of the interface screen.

The manager device 300 may receive a signal related to the user's wearing state of the HMD device 110 from the HMD device 110 and determines the wearing state of the user based on the received signal. And then, The manager device 300 may transmit an instruction for displaying guidance information guiding the wearing of the user according to the determined wearing state to the HMD device 110.

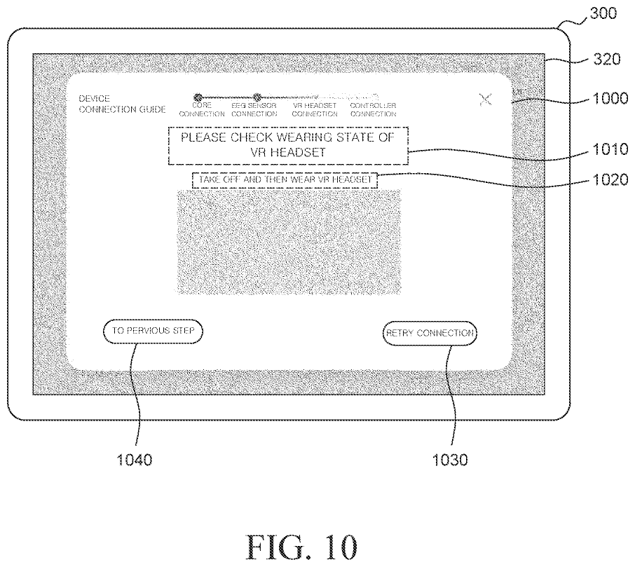

Referring to FIG. 10, the manager device 300 may check the user's wearing state of the HMD device 110 and may display a mirrored interface screen 1000 including guidance information for guiding wearing.

Here, the mirrored interface screen 1000 may include a first graphic object 1010 for instructing to check the wearing state of the HMD device 110 and a second graphic object 1020 representing guidance information for guiding the wearing. Further, the interface screen 1000 may further include a third graphic object 1030 for retrying the connection with the HMD device 110 and a fourth graphic object 1040 for proceeding to the previous step.

The manager device may support to wear the HMD device 110 by means of the interface screen.

The manager device 300 may receive a signal related to the connection state with the input device 115 from the HMD device 110 and may determines the connection state of the input device 115 based on the received signal. And then, The manager device 300 may transmit the instruction for displaying guidance information guiding the connection with the input device 115 according to the determined connection state to the HMD device 110.

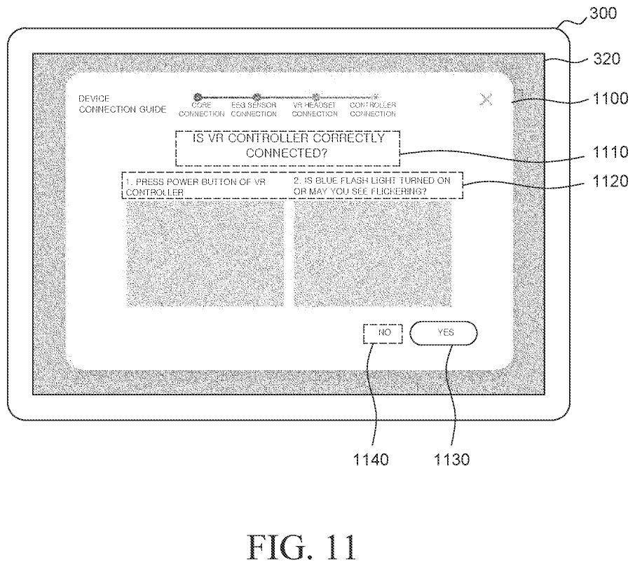

Referring to FIG. 11, the manager device 300 may display a mirrored interface screen 1100 including guidance information for guiding the connection with the input device 115.

Here, the mirrored interface screen 1100 may include a first graphic object 1110 for instructing to check the connection state of the input device 115 and a second graphic object 1120 representing guidance information for guiding the connection with the input device 115. Further, the interface screen 1100 may further include a third graphic object 1130 for inputting whether a power LED of the input device 115 is turned on and a fourth graphic object 1140 for inputting whether a power LED of the input device 115 is turned off.

The manager device may support the connection of the input device 115 by means of the interface screen.

The manager device 300 may receive a signal related to the power state of the communication connection equipment from the HMD device 110 and may determine the power state of the communication connection equipment based on the received signal. After, the manager device 300 may transmit the instruction for displaying guidance information guiding the power on/off of the communication connection equipment according to the determined power state to the HMD device 110.

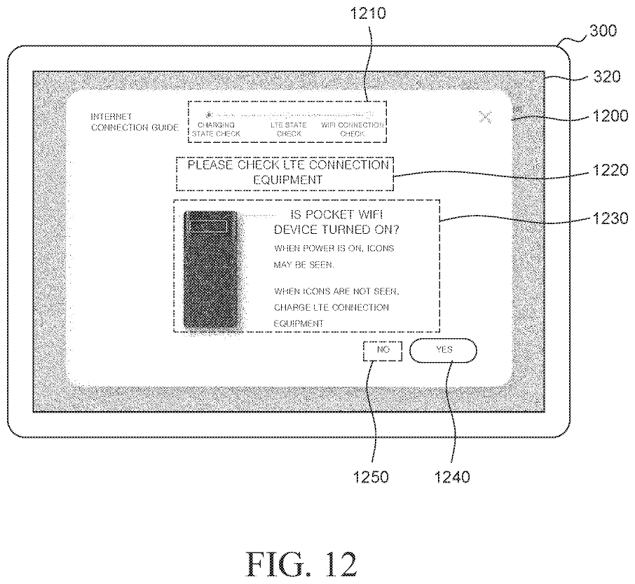

Referring to FIG. 12, the manager device 300 may display a mirrored interface screen 1200 including guidance information for guiding the power on/off of the communication connection equipment.

The mirrored interface screen 1200 may include a first graphic object 1210 representing a communication connection process, a second graphic object 1220 for instructing to check the power state of the communication connection equipment, and a third graphic object 1230 representing guidance information related to the power of the communication connection equipment. For example, the guidance information may include information guiding to check whether the power LED is turned on and information guiding the charging of the communication connection equipment, but is not limited thereto.

The interface screen 1200 may further include a fourth graphic object 1240 for inputting whether the power of the communication connection equipment is turned on and a fifth graphic object 1250 for inputting whether the power is turned off.

The manager device may support to turn on/off the power of the communication connection equipment by means of the interface screen.

The manager device 300 may receive a signal related to the communication connection state of the communication connection equipment from the HMD device 110 and may determine the communication connection state of the communication connection equipment based on the received signal. After, the manager device 300 may transmit the instruction for displaying guidance information guiding the communication connection of the communication connection equipment according to the determined communication connection state to the HMD device 110.

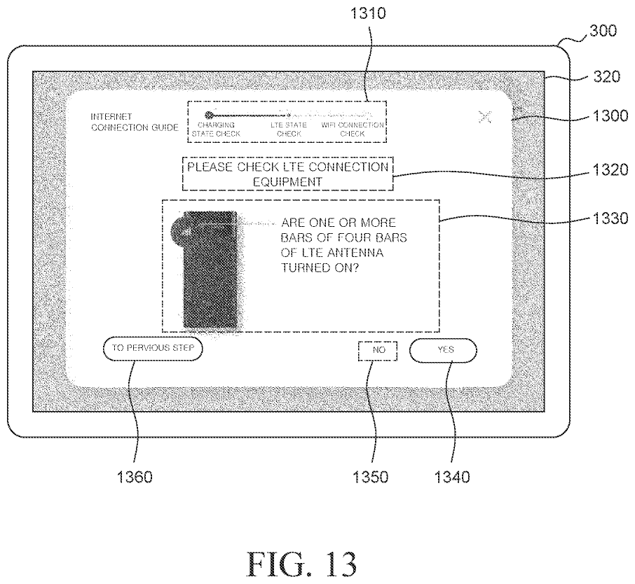

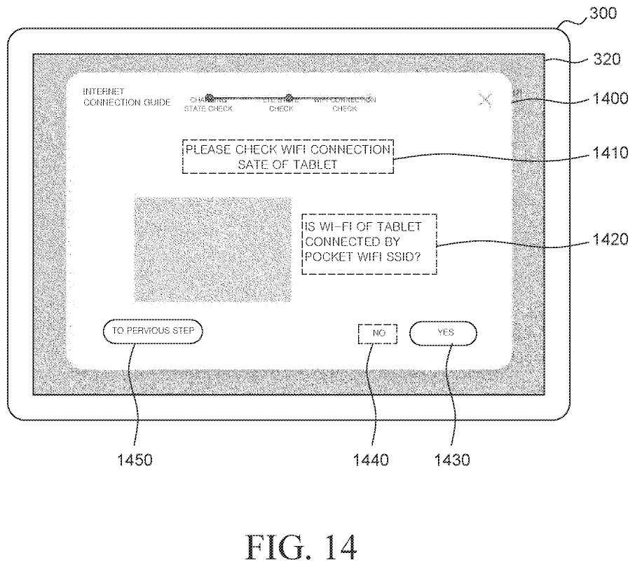

Referring to FIGS. 13 and 14, the manager device 300 may display mirrored interface screens 1300 and 1400 including guidance information for guiding the communication connection of the communication connection equipment.

Here, the mirrored interface screens 1300 and 1400 may include first graphic objects 1320 and 1410 for instructing to check the communication connection state of the communication connection equipment and second graphic object 1330 and 1420 representing guidance information for guiding the communication connection of the communication connection equipment. According to various exemplary embodiments, the mirrored interface screen 1300 may further include a graphic object 1310 corresponding to an image or an icon representing the completed communication connection process and an ongoing communication connection process, but is not limited thereto. According to various exemplary embodiments, the mirrored interface screen 1400 of FIG. 14 may also further include a similar graphic object thereto.

The interface screens 1300 and 1400 further include third graphic objects 1340 and 1430 for inputting that the communication connection equipment is normally connected, fourth graphic objects 1350 and 1440 for inputting that the communication connection equipment is not connected, and fifth graphic objects 1360 and 1450 for proceeding to a previous step.

The manager device may support the communication connection of the communication connection equipment by means of the interface screen.

According to various exemplary embodiments, when an error for the communication connection occurs, the manager device 300 may transmit instruction for displaying guidance information guiding the occurrence of the error to the HMD device 110. For example, when a communication connection speed is lower than a threshold communication connection speed that it is difficult to conduct the cognitive function test for a predetermined time or when the communication connection is disconnected, the manager device 300 may determine that the error for the communication connection occurs.

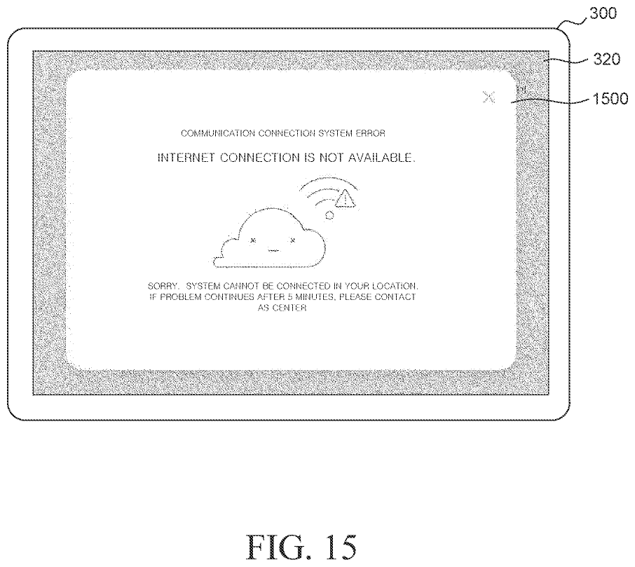

Referring to FIG. 15, the manager device 300 may display a mirrored interface screen 1500 including guidance information for guiding the error occurrence. Here, the mirrored interface screen 1500 may include a graphic object notifying that the error for the communication connection occurs and guiding a processing method according to the occurred problem.

When the device connection and the communication connection are completed, the manager device 300 may transmit an instruction to conduct the tutorial to the HMD device 110.

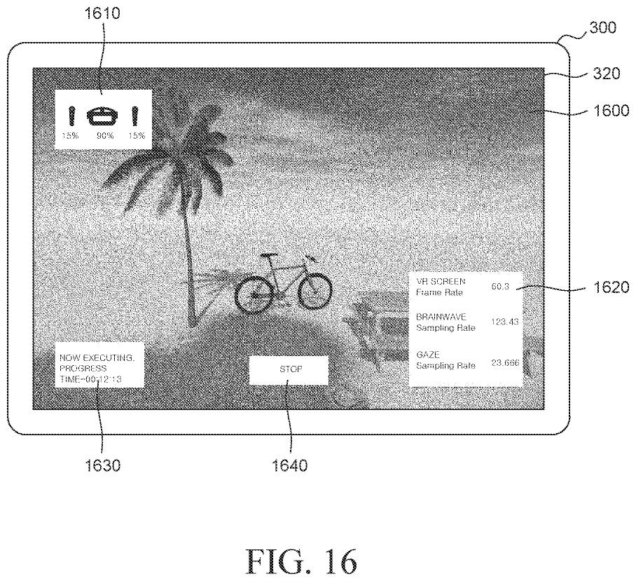

When the tutorial is conducted by the HMD device 110, the manager device 300 may display the interface screen 1600 regarding the tutorial, as illustrated in FIG. 16.

Referring to FIG. 16, the interface screen 1600 may include a first graphic object 1610 representing a charging state of the HMD device 110 and the input device 115 being connected, a second graphic object 1620 representing a frame rate of the HMD device screen, a sampling rate of the brainwave signal, and a sampling rate of the gaze signal during the tutorial, and a third graphic object 1630 representing whether the tutorial is executed and a conducted time on the mirrored interface screen of the tutorial being conducted in the HMD device 110.

By doing this, the manager monitors whether the user correctly conducts the tutorial, whether a biosignal and input data are normally acquired during the tutorial, and whether the device connection and the power connection are normally conducted during the tutorial.

When there is a problem during the tutorial, the interface screen 1600 may further include a fourth graphic object 1640 for stopping the tutorial. For example, when the fourth graphic object 1640 is selected by the manager, the manager device 300 may transmit an instruction for displaying guidance information recommending stopping the tutorial progress to the HMD device 110, and may stop the tutorial progress.

According to various exemplary embodiments, when the charging state of the HMD device 110 or the input device 115 is determined that it is difficult to conduct the tutorial or when the acquired biosignal and input data are determined that it is difficult to conduct the tutorial, the manager device 300 may transmit an instruction for displaying guidance information for recommending to stop the tutorial progress to the HMD device 110. The manager device 300 may stop the tutorial progress.

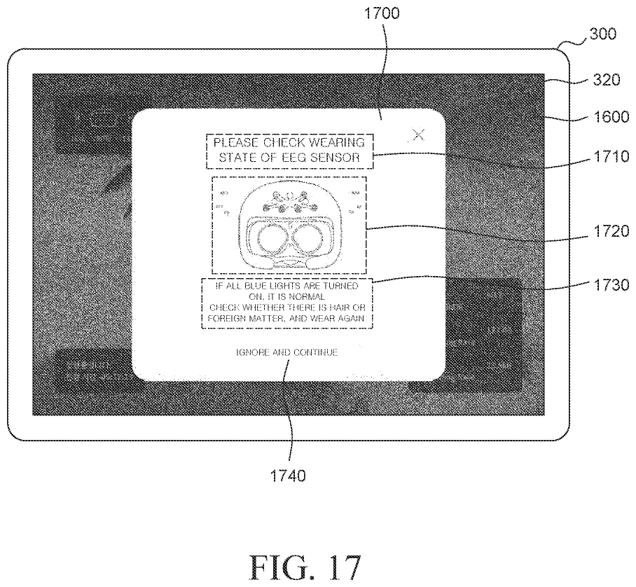

According to various exemplary embodiments, the manager device 300 may determine whether the biosignal received during the progress of the tutorial is analyzable. When it is determined that the biosignal is not analyzable, as illustrated in FIG. 17, the manager device 300 may display the interface screen 1700 including guidance information related to the sensor module or the separate sensor device.

Here, the interface screen 1700 may include a first graphic object 1710 instructing to check the wearing state of the HMD device 110 that the sensor module is installed and the wearing state of the separate sensor device, a second graphic object 1720 representing at least one sensing electrode acquiring an analyzable biosignal or a biosignal that is not analyzable, and a third graphic object representing guidance information for guiding the wearing of the HMD device 110 or the wearing of the separate sensor device. For example, the second graphic object 1720 may be an image for a bio module or a separate sensor device that represents a sensing electrode acquiring an analyzable biosignal, among at least one sensing electrode, with a first color (for example, blue) and represents a sensing electrode acquiring a non-analyzable biosignal with a second color (for example, yellow) different from the first color.

The interface screen 1700 may further include a fourth graphic object 1740 for conducting by ignoring the re-wearing of the HMD device or the separate sensor device.

The manager may support or monitor the progress of the tutorial of the user by means of the interface screen.

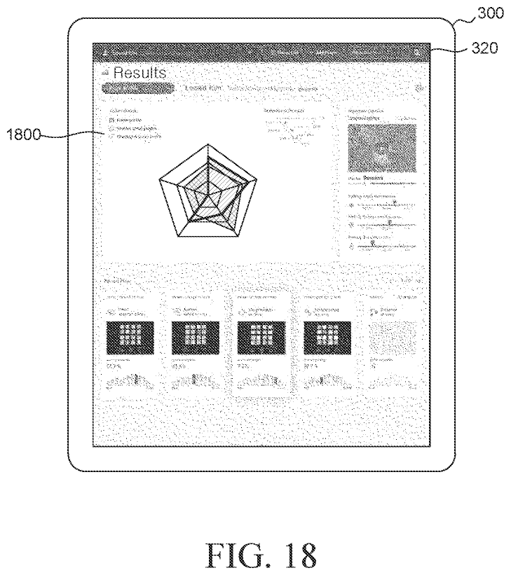

When the tutorial and the cognitive function test are completed, the manager device 300 may receive evaluation result data for the cognitive function of the user from the service providing server 130. The manager device 300 may display an interface screen 1800 representing the received evaluation result data as illustrated in FIG. 18.

Referring to FIG. 18, the interface screen 1800 may include evaluation result data provided based on the manager who supports the cognitive function test.

According to various exemplary embodiments, the evaluation result data may be provided to the HMD device 110 and the manager device 300 or provided only to the manager device 300. According to various exemplary embodiments, the evaluation result data is provided to the HMD device 110 and the manager device 300 may display the mirrored screen.

The manager may provide a result for the cognitive function test to the user by means of the interface screen and may consult the cognitive function of the user based thereon.

According to the proposed exemplary embodiment, configurations of various interface screens related to the cognitive function test program and the graphic objects are not limited to the above description and the interface screens and graphic objects may be configured in various forms.

Even though according to the proposed exemplary embodiment, the interface screen related to the cognitive function test program has been described, the interface screen related to the cognitive function training program is also provided in a similar way as described above.

As described above, according to the present invention, the user may more easily and conveniently perform the cognitive function activity with the help of the manager and the manager helps or monitors the progress of the cognitive function activity of the user to provide higher professionalism and quality diagnostic results while reducing the time and resources required for the progress of the cognitive function activity.

The device and the method according to the exemplary embodiment of the present invention may be implemented as a program command which may be executed by various computers to be recorded in a computer readable medium. The computer readable medium may include solely a program command, a data file, and a data structure or a combination thereof.

The program commands recorded in the computer readable medium may be specifically designed or constructed for the present invention or those known to those skilled in the art of a computer software to be used. Examples of the computer readable recording medium include magnetic media such as a hard disk, a floppy disk, or a magnetic tape, optical media such as a CD-ROM or a DVD, magneto-optical media such as a floptical disk, and a hardware device which is specifically configured to store and execute the program command such as a ROM, a RAM, and a flash memory. Examples of the program command include not only a machine language code which is created by a compiler but also a high level language code which may be executed by a computer using an interpreter.

The above-described hardware device may operate as one or more software modules in order to perform the operation of the present invention and vice versa.

Although the exemplary embodiments of the present invention have been described in more detail with reference to the accompanying drawings, the present invention is not limited thereto and may be embodied in many different forms without departing from the technical concept of the present invention. Therefore, the exemplary embodiments of the present invention are provided for illustrative purposes only but not intended to limit the technical concept of the present invention. The scope of the technical concept of the present invention is not limited thereto. Therefore, it should be understood that the above-described exemplary embodiments are illustrative in all aspects and do not limit the present invention. The protective scope of the present invention should be construed based on the following claims, and all the technical concepts in the equivalent scope thereof should be construed as falling within the scope of the present invention.

What is claimed is:

1. A user interface providing apparatus for supporting a cognitive function activity, the user interface providing apparatus comprising:
a communication unit configured to transmit and receive data;
a display unit configured to display data; and
a control unit configured to be operably connected to the communication unit and the display unit,
wherein the control unit is configured to connect a head-mounted display (HMD) device worn on a user for screen mirroring, receive a signal related to a wearing state of the user from the HMD device, determine a user's wearing state based on the received signal related to the wearing state, instruct the HMD device to display wearing guidance information for guiding the wearing of the user according to the determined user's wearing state, and display the mirrored cognitive function activity screen while performing the cognitive function activity by the HMD device.

2. The user interface providing apparatus for supporting a cognitive function activity of claim 1, wherein the control unit is configured to receive a signal related to an operation state of at least one sensor module installed in the HMD device from the HMD device, determine an operation state for the at least one sensor module based on the received signal related to the operation state, and instruct the HMD device to display guidance information for guiding a state of at least one sensor module according to the determined operation state.

3. The user interface providing apparatus for supporting a cognitive function activity of claim 1, wherein the control unit is configured to receive a biosignal from at least one sensor module installed in the HMD device, determine whether the received biosignal is an analyzable signal and when the biosignal is a non-analyzable signal, instruct the HMD device to display guidance information for guiding the state of the at least one sensor module.

4. The user interface providing apparatus for supporting a cognitive function activity of claim 3, wherein in order to determine whether the biosignal is an analyzable signal, the control unit is configured to use an artificial intelligence-based classification model trained to classify whether the biosignal is the analyzable signal or the non-analyzable signal by inputting the biosignal.

5. The user interface providing apparatus for supporting a cognitive function activity of claim 1, wherein the control unit is configured to instruct the HMD device to conduct a tutorial before performing the cognitive function activity and display a mirrored tutorial screen while performing the tutorial by the HMD device, and
wherein the mirrored tutorial screen includes an interface screen related to the progress of the tutorial.

6. The user interface providing apparatus for supporting a cognitive function activity of claim 5, wherein the interface screen related to the tutorial progress is an interface screen for instructing to display guidance information for guiding the tutorial progress on a tutorial screen of the HMD device.

7. The user interface providing apparatus for supporting a cognitive function activity of claim 1, wherein the control unit is configured to display a mirrored analysis result screen with regard to the cognitive function activity, and
wherein the mirrored analysis result screen includes evaluation result data provided based on a manager who supports the cognitive function activity.

8. A user interface providing method performed by a user interface providing device for supporting a cognitive function activity, comprising:
connecting a head-mounted display (HMD) device worn on a user for screen mirroring;
receiving a signal related to a wearing state of the user from the HMD device;
determining a user's wearing state based on the received signal related to the wearing state;
instructing the HMD device to display wearing guidance information for guiding the wearing of the user based on the determined user's wearing state; and
displaying a mirrored cognitive function activity screen while performing the cognitive function activity by the HMD device.

9. The user interface providing method for supporting a cognitive function activity of claim 8, further comprising the steps of:
receiving a signal related to an operation state of at least one sensor module installed in the HMD device from the HMD device;
determining an operation state for the at least one sensor module based on the received signal related to the operation state; and
instructing the HMD device to display guidance information for guiding a state of the at least one sensor module according to the determined operation state.

10. The user interface providing method for supporting a cognitive function activity of claim 8, further comprising the steps of:
receiving a biosignal from at least one sensor module installed in the HMD device;
determining whether the received biosignal is an analyzable signal; and
instructing the HMD device to display guidance information for guiding a state of the at least one sensor module when the biosignal is a non-analyzable signal.

11. The user interface providing method for supporting a cognitive function activity of claim 10, wherein in order to determine whether the biosignal is an analyzable signal, the step of determining is configured to use an artificial intelligence-based classification model trained to classify whether the biosignal is the analyzable signal or the non-analyzable signal by inputting the biosignal.

12. The user interface providing method for supporting a cognitive function activity of claim 8, further comprising the steps of:
instructing the HMD device to perform a tutorial before performing the cognitive function activity; and
displaying a mirrored tutorial screen while the HMD device performs the tutorial,
wherein the mirrored tutorial screen includes an interface screen related to the progress of the tutorial.

13. The user interface providing method for supporting a cognitive function activity of claim 12, wherein the interface screen related to the tutorial progress is an interface screen for instructing to display guidance information for guiding the tutorial progress on a tutorial screen of the HMD device.

14. The user interface providing method for supporting a cognitive function activity of claim 8, further comprising: a step of:
displaying a mirrored analysis result screen with regard to the cognitive function activity,
wherein the mirrored analysis result screen includes evaluation result data provided based on a manager who supports the cognitive function activity.
